(19) United States
(12) Patent Application Publication
(10) Pub. No.: US 2006/0177487 A1
(43) Pub. Date: Aug. 10, 2006

(54) Moisture and lotion retainer

(76) Inventor: Christine Martz, North Bellmore, NY (US)

Correspondence Address: ALFRED M. WALKER, 225 OLD COUNTRY ROAD, MELVILLE, NY, 11747-2712, US

(21) Appl. No.: 11/348,614

(22) Filed: Feb. 7, 2006

Related U.S. Application Data
(60) Provisional application No. 60/650,702, filed on Feb. 7, 2005.

(51) Int. Cl. A61K 9/70

(52) U.S. Cl. 424/443

(57) ABSTRACT

An occlusive retainer article to maintain user manually applied cosmetics, such as lotions, creams, and/or oils on human and animal skin in desired locations.

### CROSS REFERENCE TO RELATED APPLICATIONS

This application claims the benefit under 35 U.S.C. 119(e) of U.S. Provisional Application No. 60/650,702, filed Feb. 7, 2005, which is hereby incorporated by reference.

### TECHNICAL FIELD

This disclosure relates to a protective retainer or covering to be worn on skin to maintain moisturizing lotions, creams, oils or other cosmetics in a desired location.

### BACKGROUND

Topical applications of cosmetic preparations, lotions and moisturizer, electrical stimulation, collagen injections and cosmetic surgery are often used as treatments to prolong or promote youthful appearance include.

The visible signs of aging result in the appearance of wrinkles and fine lines from the effects of exposure to sun, wind, chemicals and the like. The visible signs of passage of time is reflected; wrinkles, by a slackening of tissue; a loss of cutaneous elasticity; a leathery or dry appearance; and by the yellowing of the skin which becomes duller and loses its radiance. Skin can be consistently exposed to sunlight throughout life, particularly the face and hands, may show pigmentation marks, telangiectasia and elastosis. At the histological level, skin damage from photoaging is shown in tangled, thickened, abnormal elastic fibers, decreased collagen and increased glycosaminoglycan content. The aging process results in thinning and deterioration of the skin. There may also be a reduction in cells and in blood supply, and a flattening in the junction between the dermis and epidermis. Common skin treatments are creams or lotions, which are emulsions of lipids and other components.

Common adhesive strips, such as those sold under the tradename BANDAID®, are aerated bandages held in place by adhesive substrates. The bandages cover a wound and medication applied over the wound. Because of the wound, the bandages are aerated and non-occlusive, to allow aeration of the wound during the healing process.

U.S. Pat. No. 4,411,263 of Cook describes a baby's eye shield which is adhesively applied to the skin of the face of the infant and U.S. Pat. No. 1,140,975 of Frankel describes a user-applied beauty mark comprising an adhesive patch having a decorative member thereon. Additionally, U.S. Pat. No. 6,241,998 of Muchin describes a skin-applied adhesive patch which has dermatological agents applied to the patch during manufacture for delivery of the dermatological agents from the patch to the affected area of the skin, or for exfoliation of the skin.

### OBJECTS OF THE INVENTION

It would be advantageous to maintain topical treatments on the skin as long as possible to promote skin improvement.

It is also an object to provide an occlusive retainer which retains topical substances upon the skin.

### SUMMARY

This disclosure relates to a retainer article for maintaining lotion in the desired location on human or animal skin. The retainer article can be a thin, flexible, occlusive film layer having a medically acceptable, pressure-sensitive adhesive layer and a soft material layer, such as cotton or bandage materials. Also disclosed is a method of maintaining lotion on human skin having the steps of forming a retainer article of a thin, flexible, occlusive film having a medically acceptable adhesive layer, and a soft, non-abrasive material layer; applying lotion to human skin, applying the retainer article above the lotion, and applying pressure to the film to adhere the adhesive to the skin. Alternatively, the user can apply the lotion to the soft, non-abrasive material layer of the retainer, and then the user can apply the retainer, having the user-applied lotion, to a selected region of the skin.

### BRIEF SUMMARY OF THE DRAWINGS

FIG. 1 is a perspective drawing of a retainer article designed for use between a person's eyebrows to help eliminate or reduce wrinkles in that area.

FIG. 2 is a cut-away drawing of a retainer article showing the outer film layer and the inner material layer.

FIG. 3 is a perspective drawing of a retainer article as the removable layer is taken from the adhesive layer in preparation for application over a lotion on skin.

FIG. 4 is a perspective drawing of retainer articles as they may be placed to retain lotion under a person's eyes.

FIG. 5 is a perspective drawing of retainer articles as they may be placed to retain lotion on wrinkles around a person's mouth.

FIG. 6 is a perspective drawing of the retainer article as it may be used to maintain lotion on another location of the skin, such as lotion for eczema.

FIG. 7 is a perspective drawing of the retainer article as it may be used to maintain lotion on a dry patch of skin on a person's knee.

### DESCRIPTION

This disclosure relates to an article that retains skin lotions, moisturizers, and the like on human or animal skin in the desired location. The skin lotions, moisturizers, cosmetic creams, cosmetic oils, and the like (herein after referred to collectively as “lotions”) are frequently used to moisturize the skin, soothe a sunburn, combat wrinkles and/or medicate areas with dermatological problems, such as eczema and the like. In one embodiment, the disclosed article can be used as a retainer to maintain lotions and the like in a desired location on a human face to treat wrinkle and dryness. Often these lotions are applied at night before retiring to bed, to allow the lotions to absorb into the skin. The retainer article allows the lotion to remain in the desired location in an undisturbed manner, rather than worn or rubbing away on clothing, bedding, or the users' hair.

The retainer article can be formed of a thin, flexible, occlusive film which is non-abrasive, non-reactive, and comfortable against the skin. In one embodiment, the film can be formed of a polymeric material. In another embodiment, the film can be formed of cellophane. In yet another embodiment, the film can be a plastic, or similar synthetic material, such as, for example, a thermoplastic polyurethane, urethane, polyurethane, polyethylene, and/or acrylic, which is acceptable for long term use against animal skin. In one embodiment, the film can be perforated to allow aeration of the skin. In one embodiment, the film may include ultraviolet (“UV”) protection/shielding/screen for the skin for use during the day or to protect a person's skin from UV exposure while spending time outdoors. The film material prevents lotions applied to the skin from migrating onto clothing, bedding or other undesired area while the lotion is in use.

A surface of the retainer film is a skin contact surface and may include a medically acceptable, thin, pressure-sensitive, adhesive layer, such as the adhesives used by self-sticking bandages or acrylic adhesive. Alternatively, various thermoplastic resins such as olefin based resins, ethylene vinyl acetate copolymer based resins, and elastomer based resins may also be used. For example, an ethylene methacrylate (“EMA”) based resin, an amorphous polyalpha olefin based resin, an ethylene vinyl acetate copolymer (“EVA”) based resin, anolefin based EMA blended product, a polyurethane based resin, a low density polyethylene (“LDPE”), a linear low density polyethylene (“LLDPE”) based resin, an ethylene-methylmethacrylate copolymer (“EMMA”) based resin, an elastic, non-chlorine based thermoplastic elastomer resin, other resins and the like, can be used.

The adhesive layer can be formed or deposited on the circumferential edge of the contact surface of the film for ease of placement on the desired location of skin. The adhesive may have enough tack to remain in the desired location for a number of hours, and also be removed after use without injuring the skin.

The skin contact surface may also include a soft, non-abrasive material layer, such as cotton, cotton blends, felt, gauze, bandage materials, Telfa® non-stick pads, paper pads, and the like. This material layer can form a thin, flexible layer on the contact surface of the retainer film within the circumferential edge adhesive layer. This soft material layer, however, is not necessarily co-terminus with the adhesive layer. The soft material layer maintains or assists in maintaining the lotion in its applied location. The lotion applied to the skin forms a layer between the soft material layer and the skin.

Unlike bandages used on wounds, scratches and the like, the moisture retainer does not require aeration points as a bandage would for the healing process. Additionally, the moisture retainer does not require sterility as a bandage does, although a certain level of cleanliness is preferable for contact with the skin.

The adhesive layer of the skin contact surface of the retainer article is then applied to the skin with slight pressure to adhere the adhesive layer to the skin in the desired location. The retainer may be placed over the applied lotion. The retainer may, for example, be placed under a person's eyes, on the forehead or brow line, along cheek wrinkle lines and/or any other desired location. Alternatively, the retainer may be placed on the skin of a person or animal to maintain lotions on any location on the skin.

The retainer article can have a removable, releasable, protective layer over the contact surface of the film. The protective layer maintains the material layer in a clean state before use, and prevents the adhesive layer from sticking unnecessarily to unwanted surfaces or the film itself before use. Alternatively, a removable, releasable, protective layer may be located over the adhesive layer. In yet another embodiment, the retainer article may be placed in packaging such as foil packs or blister packs for cleanliness and storage before use. The retainer articles may be shaped or formed in convenient, individual oval or generally “bean-shaped” articles. These shapes can be conveniently applied under a person's eyes and/or various locations on the face and body. Additionally, retainer may be formed in large ovals, shapes which conform to the contours of the skin of an animal or human's body and/or other shapes to be divided to a desired or suitable size according to individual needs.

Once a lotion is applied to skin, the retainer article can be placed over the lotion coated skin and adhered to the skin by applying slight pressure to an outer surface of the film above the adhesive layer. The retainer may be retained in the desired location on the skin over night, or for a time period determined by the users.

It will be understood that the retainer articles are a great advantage because the retainer hold the lotions in the desired locations to allow at least sufficient time to become effective. The retainer article assists in maintaining a youthful appearance, which is of great importance to many people, particularly in an aging population. The lotion is applied to the skin and the retainer is then placed over the area to maintain the lotion in that location. The retainer enables the lotion to be maximally effective, and hence less lotion can be used in a more cost effective manner. The retainer article also prevents the lotion from rubbing off on to clothing, bedding and the like and thereby protects the clothing and bedding. The retainer article is also conveniently disposable after use.

With respect to the drawing Figures, FIG. 1 shows occlusive horseshoe-shaped retainer article 10 applied to forehead skin region 1, above the nose and between the eyebrows, of a person, to help eliminate or reduce wrinkles in the forehead skin region 1.

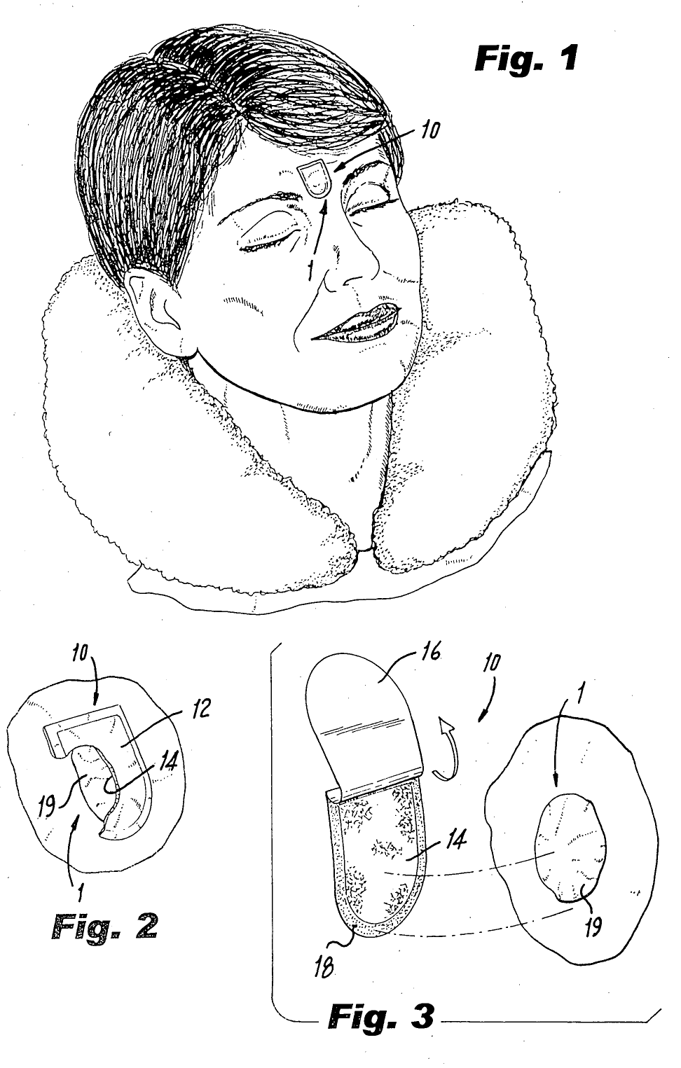

FIG. 2 shows retainer article 10 having occlusive outer film layer 12 and the inner material layer 14 retaining dosage dab 19 of a moisturizer lotion 19 or other similar material.

FIG. 3 shows retainer article 10 as removable layer 16 is taken from the adhesive layer 18 in preparation for application over lotion 19 on the skin of a person.

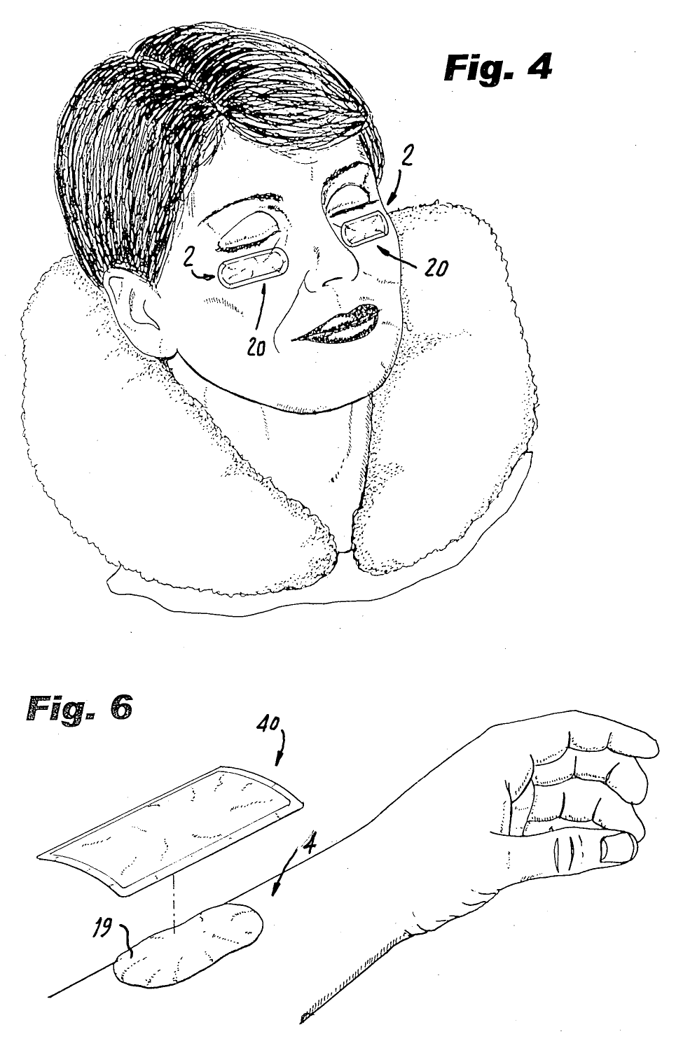

FIG. 4 shows oval shaped strip-shaped retainer articles 20 as they may be placed to retain lotion 19 in the skin regions 2 under a person's eyes.

FIG. 5 shows crescent shaped retainer articles 30 as they may be placed to retain lotion on wrinkles in the region 3 around a person's mouth.

FIG. 6 shows rectangular occlusive retainer article 40 as it may be used to maintain lotion 19 on a forearm skin region 4 of the skin of a person, such as a lotion for eczema.

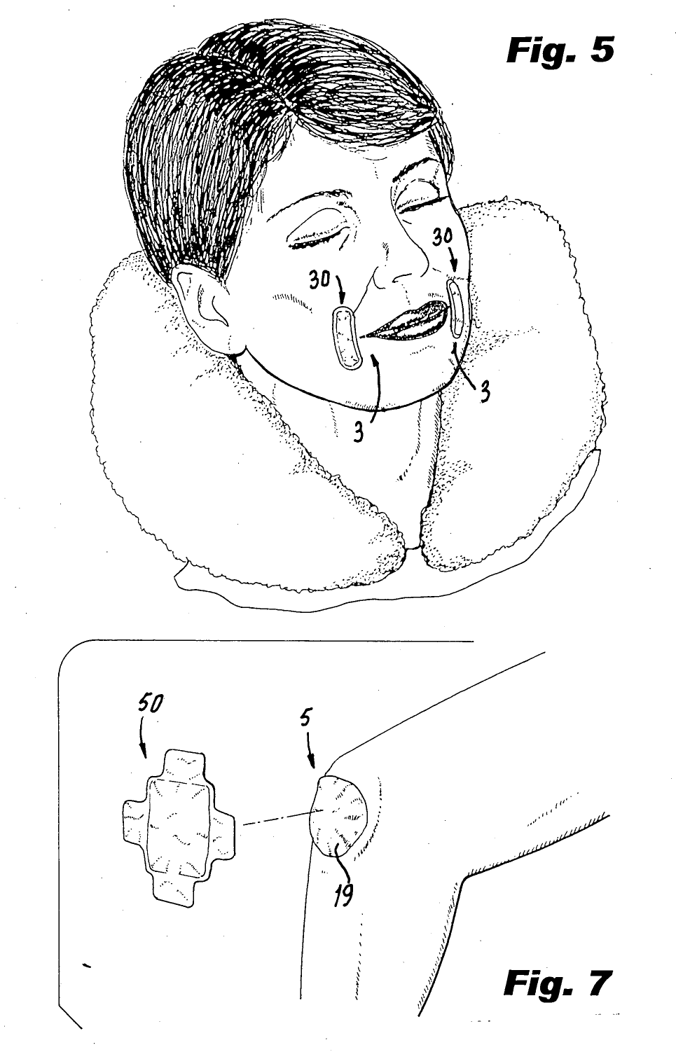

FIG. 7 shows flat dual terraced occlusive retainer article 50 as it may be used to maintain lotion 19 on a dry patch of skin in skin region 5 on a person's knee.

As noted before, the above drawings show that the occlusive retainer articles may be formed in various shapes, such as large ovals, bean shapes or other geometric shapes which conform to the contours of the skin of an animal or human's body and/or other shapes to be divided to a desired or suitable size according to individual needs.

The above examples are intended to illustrate but not limit the invention. While they are typical of those that might be used, other procedures known to those skilled in the art may alternatively be used.

What is claimed is:

1. A retainer article for maintaining a user manually applied cosmetic in the desired location upon an animal or person comprising: a thin, flexible, occlusive non-aerated film layer; an adhesive layer and a soft material layer.

2. A retainer article of claim 1, wherein the film layer is a plastic.

3. A retainer article of claim 1, wherein the film layer is polymeric.

4. A retainer article of claim 1, wherein the film layer is cellophane.

5. A retainer article of claim 1, wherein the film layer is thermoplastic polyurethane.

6. A retainer article of claim 1, wherein the film layer includes an ultraviolet shield.

7. A retainer article of claim 1, wherein the film layer has a skin contact surface.

8. A retainer article of claim 7, wherein the skin contact surface has an adhesive layer.

9. A retainer article of claim 8, wherein the adhesive layer formed on a circumferential edge of the film layer.

10. A retainer article of claim 9, wherein the adhesive layer is medically acceptable.

11. A retainer article of claim 9, wherein the adhesive layer is acrylic.

12. A retainer article of claim 9, wherein the adhesive layer is a resin.

13. A retainer article of claim 9, wherein the adhesive layer is pressure-sensitive.

14. A retainer article of claim 13, wherein the soft material layer is selected from the group consisting essentially of cotton, cotton blends, felt, gauze, bandage materials, non-stick pads and combinations thereof.

15. A retainer article of claim 14, wherein the soft material layer is located on the skin contact surface.

16. A retainer article of claim 15, wherein the retainer article has a geometric shape which conforms to the contours of an animal's or person's skin.

17. A retainer article as in claim 16, wherein said geometric shape is selected from the group consisting of an oval, a strip, a crescent, a rectangle, a bean shape, a flat terraced pad or a horseshoe.

18. A retainer article as in claim 1 wherein said cosmetic is selected from the group consisting of a lotion, a cream, an oil and/or combinations thereof.

19. A method of retaining a user manually applied cosmetic on human skin comprising the steps of forming a retainer article of a thin, flexible, non-aerated occlusive film having a medically acceptable adhesive layer, and a soft, non-abrasive material layer; manually applying a cosmetic to human skin, applying the retainer article above said cosmetic, wherein said retaining article is dimensioned to conform to the contours of the location of the skin to which the cosmetic is manually applied, and applying pressure to the film to adhere the adhesive to the skin.
